(19) United States
(12) Patent Application Publication
(10) Pub. No.: US 2004/0219184 A1
(43) Pub. Date: Nov. 4, 2004

(54) Growth of large patterned arrays of neurons on CCD chips using plasma deposition methods

(75) Inventors: Ian G. Brown, Berkeley, CA (US); Eleanor A. Blakely, Oakland, CA (US); Othon R. Monteiro, Oakland, CA (US); James E. Galvin, Emeryville, CA (US); Kathleen A. Bjornstad, Oakland, CA (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(73) Assignee: The Regents of the University of California, Oakland, CA

(21) Appl. No.: 10/809,269

(22) Filed: Mar. 24, 2004

Related U.S. Application Data
(60) Provisional application No. 60/457,760, filed on Mar. 25, 2003.

(51) Int. Cl. C12N 5/08; H01L 35/24

(52) U.S. Cl. 424/423; 435/368; 257/40

(57) ABSTRACT

Neuroelectronic devices are disclosed which use charge-coupled detector array devices to stimulate, monitor and record neural network activity and response. The method and apparatus described herein uses vacuum-arc-plasma based methods of surface modification as a tool for forming large patterned neuronal arrays on substrates. The basic device features a charge coupled detector device array (CCD) having a thin protective film over the CCD, a thin patterned film to promote neuron growth, and an insulator. Methods for stimulating and monitoring stimulated and spontaneous electrical activity in individual neurons of the array can be carried out by the apparatus described.

### CROSS-REFERENCE TO RELATED APPLICATIONS

This application claims priority from U.S. Provisional Patent Application No. 60/457,760, filed on Mar. 25, 2003, which is hereby incorporated by reference in its entirety.

### STATEMENT OF GOVERNMENTAL SUPPORT

This invention was made during work supported by U.S. Department of Energy under Contract No. DE-AC03-76SF00098. The government has certain rights in this invention.

### BACKGROUND OF THE INVENTION

1. Field of the Invention

The present invention relates to methods of cell culture on patterned surfaces and the formation of those surfaces.

2. Related Art

The study of the functional unit of the nervous system, the neuron, has been an active field of investigation for many years, both at the single-cell level, in vivo and in vitro, and at the level of large numbers of interconnected neurons, for example, within the human brain (Vaudry D., Stork P. J. S., Lazarovici P. and Eiden L. E. (2002) Science 296, 1648). The behavior of individual neurons has been studied using microelectrodes to monitor the electrical signals and action potentials generated within the neuron and along its dendrites (the branch-like arms that carry signals toward the neuron cell body where they are processed) and axons (the long “tail” that carries the neuron output signal to other cells).

The electrical behavior of individual neurons has been well studied and is reasonably well understood. The behavior of large networks of neurons, however, is not at all well understood. While techniques such as magnetic resonance imaging and positron emission tomography have provided insight into the location of neural activity within the brain, the minimum resolvable volume (˜1 mm³) contains ˜10³-10⁴ neurons.

In order to learn the details of how large systems of neurons communicate, there is a need for methods for growing networks of large numbers of live neurons whose dendrite and axon connections can be controlled in pre-determined ways, and secondly, to develop means for stimulating and monitoring excitation of individual neurons. Success with this challenge will be of great importance to our understanding of the working of the human brain and peripheral nervous system, and to novel kinds of computer architecture. Large in vitro networks could show, for example, the emergence of stable patterns of activity, and could lead to an understanding of how groups of neurons learn after repeated stimulation.

To systematically explore the electrical characteristics of large numbers of associating neurons, however, techniques must first be developed for forming 2-dimensional patterned arrays of large numbers of neurons. All of the parameters of the patterning should be under the control of and determined by the experimenter, including the geometry of the pattern, the line width, and the pattern size (number and density of neurons). The subsequent step is to discover and develop methods for monitoring the electrical activity throughout the array.

There has been good progress in the growth of random, non-patterned monolayer neural cultures in which dissociated neurons grow, extend processes, form synapses and create neural networks. Several approaches to patterning have been explored, including mechanical fabrication of troughs and ridges (Miller C., Shanks H., Witt A., Rutkowski G. and Mallapragada S. (2001) Biomaterials 22, 1263.), laser micromachining (Corey J. M., Wheeler B. C. and Brewer G. J. (1991) J. Neurosci. Res. 30, 300), surface photochemical methods (Hickman J. J., Bhatia S. K., Quong J. N., Shoen P., Stenger D. A., Pike C. J. and Cotman C. W. (1994) J. Vac. Sci. Tech. A12, 607), photoresist methods, among others. See also Stenger D. A. and McKenna T. M. (1994). Enabling Technologies for Cultured Neural Networks. Academic Press, San Diego.

Methods for monitoring the electrical activity of a small number of cells simultaneously have been developed, mostly making use of extracellular recording of the action potentials with extracellular microcircuit electrode arrays. However, many of the methods so far developed involve the use of intercellular microelectrodes. The neurons are impaled by micron-size electrodes which penetrate the cell wall. Cell death results from trauma usually within a few hours of electrode insertion. These methods are further limited in that they cannot be extended to very large arrays. Thus, it has not been possible to-date to adequately detect the action potential spatial geography and temporal history of large arrays.

Therefore, it is a goal of the invention to provide (i) methods for growing networks of live neurons on patterned substrates, and (ii) neuroelectronic devices and methods for stimulating and monitoring action potentials in individual neurons of the array. The method and apparatus described herein uses vacuum-arc-plasma based methods of surface modification as a tool for forming large patterned neuronal arrays on substrates. In a preferred embodiment, detector arrays, such as a charge-coupled device (CCD) arrays, record the neuron action potentials and activity for spatial and temporal analysis and detection.

### BRIEF SUMMARY OF THE INVENTION

The present invention provides a neuron chip platform 100 comprising a charge coupled device (CCD), a thin protective film over the CCD array, a thin patterned film to promote neuron growth, and an insulator. The CCD first is covered by a thin protective film of a transparent material to protect the CCD from corrosion or electrolysis by prolonged contact with the cell culturing layer, cell medium or cells. This protective film should be from 100 Å to 1000 Å, preferably about 500 Å to permit close contact by the cells with the CCD and allow detection of electrostatic effects due to cell activity. A patterned film, preferably comprised of diamond-like carbon is then applied to the protective film. This film should be about 100 to 300 Angstroms, preferably less than 150 Angstroms.

The neuron chip platform is further comprised of a cell culturing layer over the patterned film to promote and maintain prolonged neuron or cell growth on the platform. The cell culturing layer 40 is preferably Collagen Type I, Collagen Type IV, laminin, extracellular basement membrane proteins or combinations thereof.

In one aspect of the invention, the neuron chip platform is detachable, connected to a stimulation signal supply means and a signal or image processing means. Each neuron chip platform can be placed inside an ordinary incubator for cell culture and then taken out from the incubator and reconnected to the stimulation signal supply means and the signal or image processing means.

In a second aspect of the invention, the protective film is a single or composite layer or multiply layered film having the properties of sufficient transparency, chemical inertness and impermeability. In some embodiments, a microelectrode or conducting track is embedded in the multiply layered protective film.

In another aspect of the invention, the neuron chip platform is further comprised of a means to maintain an environment for culture of the cells on said platform to enable long-term measurement and growth. This environmental maintenance means would be comprised of a temperature adjustment means for maintaining a constant temperature, a means for circulating a culture solution, a means for supplying a mixed gas of air and carbon dioxide (e.g., CO₂ 5%), and a covering means to keep the cells enclosed on the platform.

The invention further provides a cell potential measurement apparatus 200 comprising (A) a neuron chip platform provided with a charge coupled device (CCD), a thin protective film over the CCD, a thin patterned film to promote neuron growth, and an insulator; (B) an illumination source, (C) a stimulation signal supply means for providing electrical stimulation to the cells; and (D) a signal or image processing means to be connected to the CCD for processing an output signal or image arising from electrical physiological activities of the cells.

In another aspect the invention also provides a method for non-invasive detecting and monitoring live networks of neurons comprising the steps of (a) providing a cell potential measurement apparatus; (b) adding a cell culturing layer seeded with neurons; (c) allowing the neurons to develop neurite extensions and dendritic connections; (d) providing electrical or environmental stimulation to the neurons; (e) detecting and recording the neurons response via the CCD; and (f) analyzing said neuron response using a signal or image processing means.

### BRIEF DESCRIPTION OF THE DRAWINGS

FIG. 1 is a cross-sectional view of the neuron chip platform.

FIG. 2 is a schematic showing the preferred embodiment of the apparatus, wherein FIG. 2A shows various assembly stages; and FIG. 2B shows the apparatus with signaling and measurements devices attached.

FIG. 3 is a schematic of a filtered vacuum arc plasma gun system used in making the present device.

FIG. 4 is a photograph showing selective neuron growth on diamond-like carbon-coated glass slide.

FIG. 5 is a photograph showing selective PC-12 neuron growth on collagen-coated, DLC-plasma-processed surface. A delicate neurite growth develops on the DLC-treated region (FIG. 5A; left-hand photo), which develops into a dense and prolific neuron field (FIG. 5B; right-hand photo). (Scale: the width of each photograph is about 300 microns).

FIG. 6 is a set of photographs showing patterned growth of neurons on diamond-like carbon to form “LBNL” (in negative).

### DETAILED DESCRIPTION OF THE PREFERRED EMBODIMENT

While techniques such as magnetic resonance imaging and positron emission tomography have provided insight into the location of neural activity within the brain, the minimum resolvable volume (˜1 mm³) contains ˜10³-10⁴ neurons. In order to learn the details of how large systems of neurons communicate, there is a need to develop a means and methods for growing networks of large numbers of live neurons whose dendrite and axon connections can be controlled in pre-determined ways, and to develop means for stimulating and monitoring excitation. Success with this challenge will be of great importance to the understanding of the working of the human brain and peripheral nervous system, and to novel kinds of computer architecture. Large in vitro networks could show, for example, the emergence of stable patterns of activity, and could lead to an understanding of how groups of neurons learn after repeated stimulation.

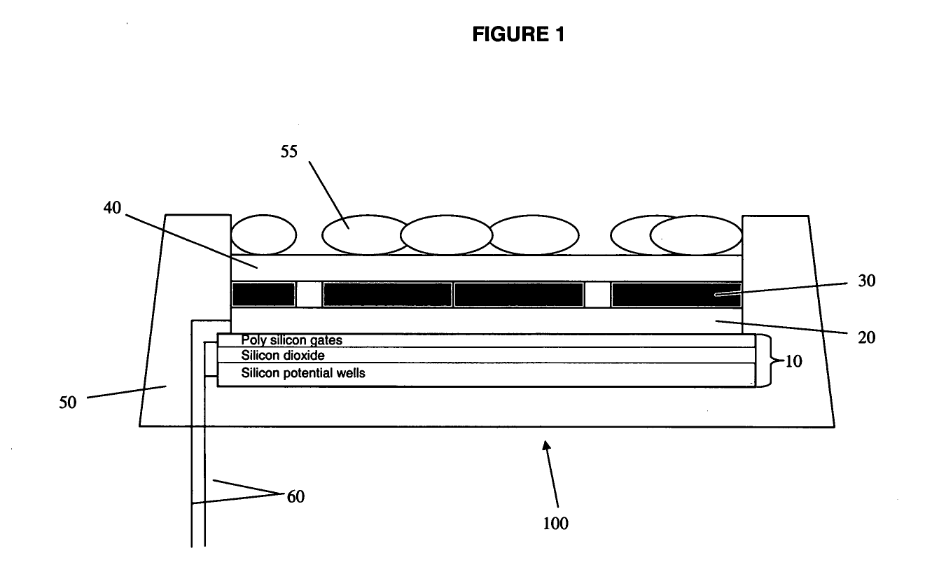

Referring to FIG. 1, the invention provides a neuron chip platform 100 comprising a charge coupled device (CCD) 10, a thin protective film 20 over the CCD array, a thin patterned film 30 to promote neuron growth, and an insulator 50 which can act as a barrier to protect the CCD circuitry and the electrical connection means 60.

The neuron chip platform is made by first providing a CCD comprising a standard pixel array 10. A protective film 20 is deposited onto the CCD array 10 using plasma deposition with a filtered vacuum arc system to a thickness of about 100 to 1000 Å. The CCD circuitry and pixel array are protected by an insulator 50 to seal and insulate from corrosion and electrolysis by the cell culturing medium and forming a neuron chip platform. Then a patterned film 30 of about 100 to 300 Å diamond-like carbon (DLC) film is then deposited on the protective film 20 according to the desired pattern of neuron growth. It is preferred that the film depositions are done under conditions which would permit the deposition of films 20 and 30 that are sufficiently free of imperfections to prevent corrosion or electrolysis of the CCD. A continuous cell culturing layer 40 of Type I Collagen or an extracellular matrix protein is then added to the top of the patterned film and seeded with neuron cells 55.

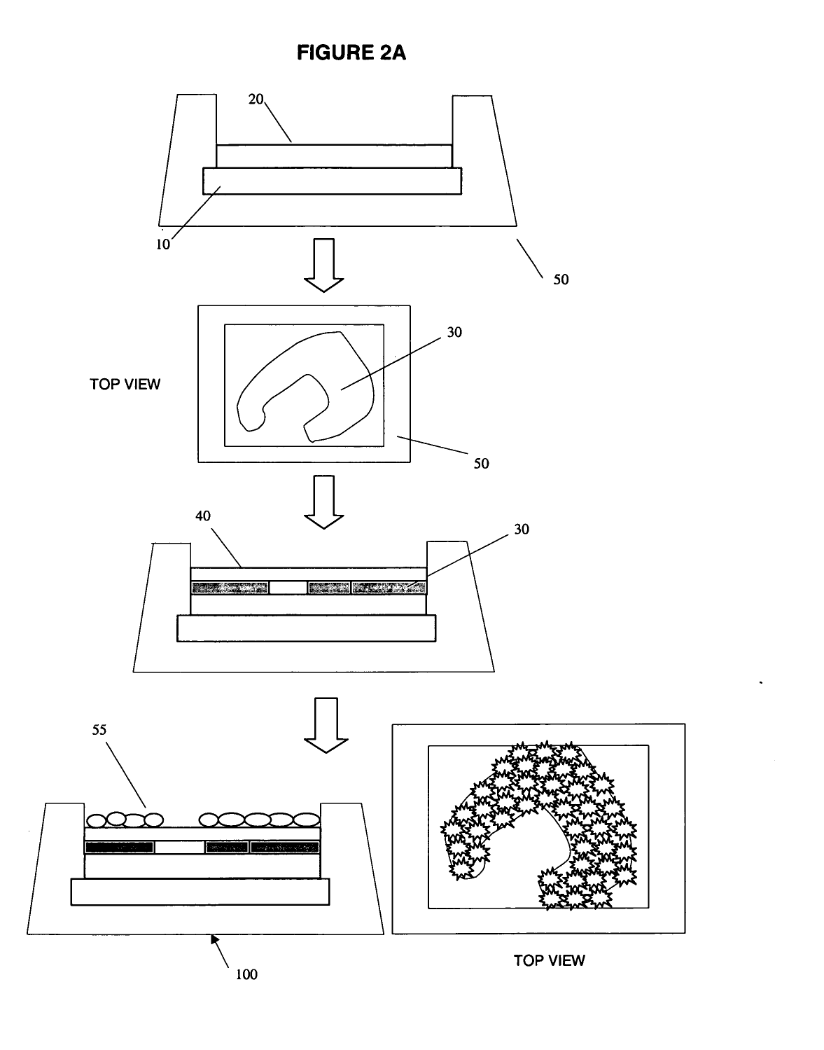

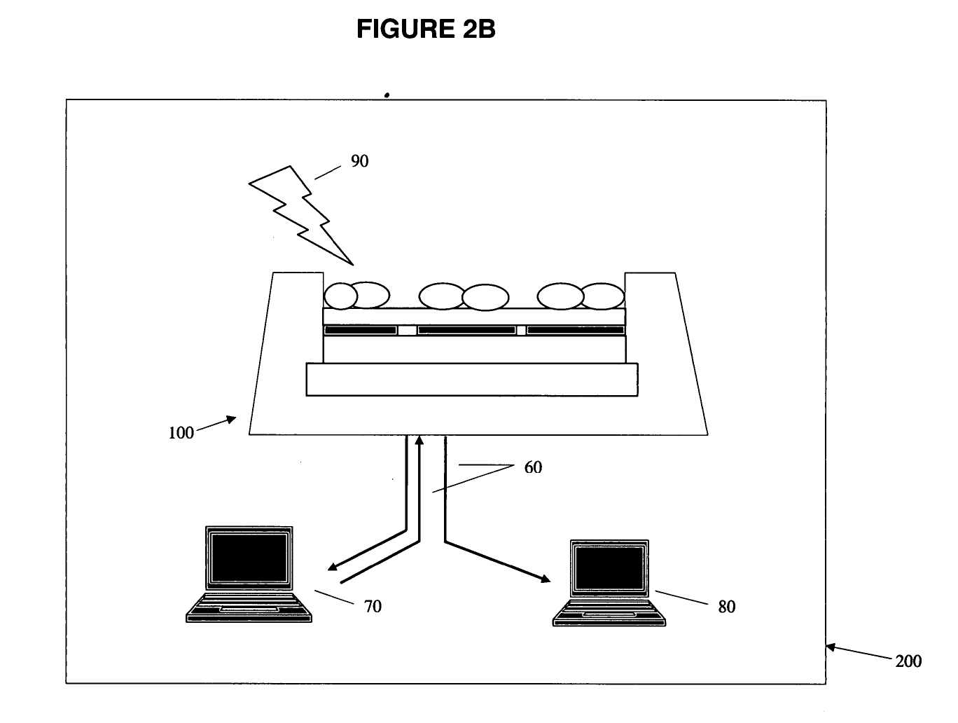

Referring to FIG. 2B, the invention further provides a cell potential measurement apparatus 200 comprising (A) a neuron chip platform 100 provided with a charge coupled device detector (CCD) array 10, a thin protective film 20 over the CCD array, a thin patterned film 30 to promote neuron growth, and an insulator 50 which can act as a barrier to protect the electrical connection means for providing an electrical signal to the CCD and for leading out an electrical signal from the microelectrodes; (B) an illumination source 90, (C) a stimulation signal supply means 70 to be connected to the electrical connection means 60 of the neuron chip platform for providing electrical stimulation to the cells; and (D) a signal or image processing means 80 to be connected to the electrical connection means 60 of the neuron chip platform 100 for processing an output signal or image arising from electrical physiological activities of the cells.

In a preferred embodiment, the neuron chip platform 100 is detachable from the stimulation signal supply means 70 and the signal or image processing means 80. Each neuron chip platform 100 can be placed inside an ordinary incubator for cell culture and then taken out from the incubator and reconnected to the stimulation signal supply means and the signal or image processing means.

In a preferred embodiment, the neuron chip platform 100 is further comprised of a means to maintain an environment for culture of the cells on said platform to enable long-term measurement and growth. This environmental maintenance means would be comprised of a temperature adjustment means for maintaining a constant temperature, a means for circulating a culture solution, a means for supplying a mixed gas of air and carbon dioxide (e.g., CO₂ 5%), and a covering means to keep the cells enclosed on the platform.

A. Charge-Coupled Detector (CCD) 10

The present invention comprises a charge coupled detector device (CCD) in electrical contact with a large array of interconnected cells. In the general embodiment of the neuron chip platform 100, any conventional CCD device can be used. CCDs are available in sizes ranging from ⅓ of an inch through 1″×1½″ having a range of pixels from 380,000 pixels to about 11 million pixels, with pixel sizes generally ranging from about 6 microns to about 13 microns.

The invention relies on well-known CCD array architecture and technology. In general, any CCD can be used. The typical CCD 10 is depicted in FIG. 1, having a typical base layer of silicon with potential wells, a layer of silicon dioxide, and a top layer of poly silicon gates. When exposed to photons or electrical charge, an absorbed photon creates a hole-electron pair in the silicon. The holes are lost to the base layer, and the electrons are trapped or stored temporarily in potential wells created by electrostatic fields from an array of conductors deposited on the surface of the chip. The potential wells are formed in a grid, typically 640 by 480, where each well might be 10 microns across.

For normal CCD operation, an image formed by cellular activity is focused on the chip, and the wells fill with electrons according to the illumination or electrical activity on each well. Several methods of terminating the exposure are used. For example, in the Sony ICX038DLA CCD chip, the CCD chip used in a specific embodiment, the wells are moved under opaque stripes, by changing the voltages on the conductor pattern. Again, by changing the conductor pattern voltages, the wells are moved, bucket brigade fashion, one by one to the output amplifier of the chip, thereby producing a video signal.

The present invention uses the ability of a CCD to act as an electrostatic pick-up device. Thus, the electrostatic changes brought about by a single cell propagating a nerve signal through membrane depolarization can be measured and recorded in real time. Furthermore, the nerve signal can be mapped across an area when propagated by a small number to large networks on the order of 10⁴ to 10⁶ of interconnected nerve cells that have been cultured on top of the CCD. Electrical propagation among other types of cells (e.g. cardiac, smooth or striated muscle) could also be studied. In order to directly transmit the above-described electrical changes, it is important that the CCD be in electrical contact with the neuronal culture. This is accomplished by the use of thin films applied to the CCD which are described infra.

The CCD 10 should have pixels with dimensions comparable to the size of the neurons grown on the platform. The actual “soma” or cell body of neurons can vary by species and part of the body and even which part of the brain, from which it is taken. Furthermore, the length of the axons can be very long. Thus, the size of one pixel is preferably on the order of the size of the neuronal cell to be cultured in order to detect the signal from each individual cell in the population. For signal magnification purposes, it may even be preferable that the pixel size be on the order of two pixels per one neuronal cell to be cultured. However in some embodiments, larger pixel sizes can also be used to monitor electrical properties among neuronal arrays, that is, along a neuronal path.

Therefore, it is contemplated that preferred embodiments will use CCD having the smallest pixels such as those in 1/3 inch video chips, or those used in inexpensive video cameras, such as security cameras. In a preferred embodiment, the CCD 10 should have a relatively small pixel size, preferably a pixel size of about 6 microns square (6×6) to about 15 microns square, preferably less than 7 microns square because the preferred neurons have a cell diameter that can range from 10-25 microns up to several fold larger. In a preferred embodiment, the CCD pixel array would also be a low light detector to record very low light images such as those from fluorescence. Also, in a preferred embodiment, the CCD would exhibit a high signal-to-noise ratio, low dark current, high sensitivity and good quantum efficiency.

In a specific embodiment, the CCD used is an interline CCD solid-state image sensor suitable for EIA black-and-white video cameras with a diagonal 8 mm (Type ½) system, with a chip size of 7.95 mm×6.45 mm, unit cell size of 8.4 μm×9.8 μm and 380,000 total effective pixels.

The CCD 10 further comprises standard electronics, including but not limited to silicon gates, a field period readout system, an electronic shutter with variable charge-storage, pins, socket pins, and wires, and the package. The package is the carrier into which the manufacturer typically installs the CCD pixel array and the standard electronics.

Most commercial CCD chips that are sold have a glass cover and a thick plastic polymer layer covering the CCD. To practice this invention, the glass cover and the plastic polymer layer should be first removed in order to keep the cells in close proximity to the CCD. It is often difficult to remove the plastic layer therefore it is preferred that the CCDs used in this invention be obtained without the glass cover or the plastic polymer layer. One such CCD that has no glass cover or polymer layer is the KODAK® KAI-2001 chip, which has no coverglass or microlenses, and can be obtained with evaluation board. The KODAK® KAI-2001 CCD chip has a pixel size 7.4 μm square, 1600×1200 pixels (1.9 Mpixel), active area 11.8 mm×8.9 mm, and is a color (RGB) detector array. Thus in a preferred embodiment, a CCD such as the KODAK® KAI-2001 CCD or an equivalent would be preferred because it has no coverglass or microlenses to interfere with deposition of thin films on the CCD, which are described infra.

B. Protective Film 20 over CCD Array 10

The main purpose of the protective film 20 is to protect the CCD 10 from contamination, corrosion and electrolysis due to the lack of or removed plastic cover. The protective film 20 must be inert and impermeable for extended periods of time despite being in contact with the cell culturing layer 40 and cell medium which can consist of salts, sugars and other cellular factors. Another concern is that the protective film maintains close contact with the patterned film to minimize the possibility of paths for leaks at the protective film/patterned film interface. Other considerations in making a preferred protective film 20 is the stress levels of the film because a more compressed film will be less prone to pores and is less permeable to liquids.

The protective film 20 is preferably a thin film of alumina, silica, aluminum silicate, titanium oxide, tantalum oxide, silicon dioxide and combinations thereof, or similar film that is optically transparent, chemically inert and impermeable. In other embodiments, the film 20 is a composite layer or multiply layered film having the same properties of sufficient transparency, chemical inertness and impermeability. These composite or multiply layered films should consist primarily of combinations of different oxides and/or nitrides, including but not limited to, silicon dioxide, aluminum oxide, titanium oxide, tantalum oxide, silicon nitride, aluminum nitride, titanium nitride and tantalum nitride, and can also be comprised of various materials including but not limited to, C, Mg, Ti, Pd, Ta, Ir, Pt and Au, and combinations thereof.

In a specific embodiment, the layered protective film 20 will also have microelectrodes and conducting tracks embedded or buried in the layered film. Burying the microelectrodes and conducting tracks will help maintain film integrity and minimize intrinsic stresses that may arise from cracking, delamination or exposure to corrosive liquids. See G. Schmitt et al., “Passivation and corrosion of microelectrode arrays,” Electrochimica Acta 44 (1999) 3865-3883, which is hereby incorporated by reference. The microelectrodes and conducting tracks can be made of silver, platinum, gold, titanium, carbon, silicon or other metal suitable for use as a microelectrode or conducting track. The pattern and methods for forming the microelectrode array can be similar to that as described in Strong, T. et al., “A microelectrode array for real-time neurochemical and neuroelectrical recording in vitro,” Sensors and Actuators A 91 (2001) 357-362, which is hereby incorporated by reference.

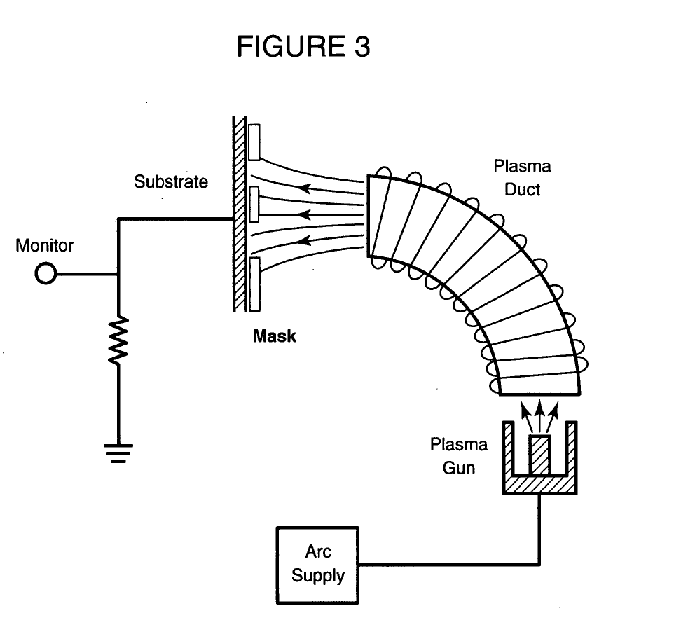

The protective film 20 should be from 100 Å to 1000 Å in thickness, preferably about 500 Å, to permit the CCD detector array close contact to detect electrostatic effects due to the cells. In a preferred embodiment, the protective film 20 is preferably made through plasma deposition with a filtered vacuum arc plasma gun system as shown in FIG. 3, discussed below.

C. Patterned Film 30 to Promote Patterned Neuron Growth

It was found that deposition of patterned films of diamond-like carbon promote patterned neuron growth. The term DLC as used herein has its standard meaning, i.e. a hard, amorphous film with a significant fraction of Sp³-hybridized carbon atoms and which can contain a significant amount of hydrogen. The films may be fully amorphous or contain diamond crystallites. In a preferred embodiment, the carbon film deposition results in the film material formed being a high quality, hydrogen-free, diamond-like carbon (DLC), not amorphous carbon or graphite.

The preferred diamond-like carbon films are strong and stable, and remain intact on a substrate for periods of at least 2 months, thus providing the basis for a substrate useful for prolonged study of neuron activity and the ability to create neuron arrays. The preferred DLC thickness for patterned film 30 to accomplish neuronal patterning is approximately 100 Å to about 300 Å, more preferably about 100 Å to about 150 Å, even more preferably about 100 Å, to result in high neuron growth contrast, or the ratio of neuron density on plasma-treated regions to neuron density on untreated regions. Neuron proliferation and the elaboration of dendrites and axons after the addition of nerve growth factor both showed excellent contrast, with prolific growth and differentiation on the DLC treated surfaces and very low growth on the untreated surfaces.

Lithographic masks to make the patterned films 30 can be created to allow subsequent proliferation and/or differentiation of the neurons to form desired patterns. Alternatively conventional photoresist and photolithography techniques can be used to form the desired pattern.

The patterning of film 30 is designed to control the connections between neuronal cells in predetermined ways. The material used in patterned film 30 is designed to facilitate neuronal attachment and growth, so that the areas of the device lacking patterned film 30 have essentially no neuronal growth. Accordingly, a neuronal body may be selected for engagement by a dendrite at a pre-determined distance from the center of the neuron by virtue of the arrangement of patterned film 30. The patterned film 30 may also be formed of an electrically conductive metal to be useful for delivering an electrical signal to a pre-selected neuron.

Patterning neural growth will also enable the observation and CCD detection of neuron activity both optically and electrically. This could permit possible communication with individual neurons of the array via both optical and electrical methods. To facilitate such communication, the patterning lines are preferably optically transparent and optionally either electrically conducting or insulating.

It is also known that the location of neuron cell attachment and the extension region of neurite outgrowths can be controlled using patterned plasma deposition into polystyrene culture dishes. Tsuji, H., Sasaki, H., Sato, H., Gotoh, Y., and Ishikawa, J, Nucl. Instrum. Meth. Phys. Res. B (in press); Tsuji, H., Sato, H., Ikeda, S., Ikemura, S., Gotoh, Y. and Ishikawa, J., Nucl. Instrum. Meth. Phys. Res. B, 148:1136 (1999). Combining this method of controlling neurite outgrowth with the method described herein of patterning neuron growth offers a promising approach to forming artificially designed neural networks in cell culture in vitro.

Good results in directed neuron growth were obtained for the case of plasma deposition of carbon to form a diamond-like carbon film of thickness about 100 Å.

D. Film Deposition

The present protective film 20 and patterned film 30 may be applied to the CCD chip 10 in various ways. These thin films allow the cells to grow in very close proximity to the CCD 10 for accurate measurement and monitoring of spontaneous or stimulated action potential activity of individual cells. The protective film 20 and the patterned films 30 can be made through metal ion implantation using a vacuum arc ion source, and plasma deposition with a filtered vacuum arc system. Lithographic masks, or conventional lithographic techniques as used for semiconductor processing, can be used to allow subsequent proliferation and/or differentiation of the neurons to form desired patterns, including but not limited to, spinning a photoresist, exposing the desired pattern, etching the pattern, or depositing the film and lift-off.

The use of metal ion implantation using a vacuum arc ion source system, and plasma deposition with a filtered vacuum arc plasma gun system, as a means of forming regions of selective neuronal attachment on surfaces was investigated it was found that plasma deposition is preferably used to deposit films as ion implantation resulted in films that poorly promoted neuron growth.

The films are preferably more compressed films because they reduce permeability to a liquid and are less prone to have pores which result in delamination. It is also preferred that the film deposition be performed in a clean room to minimize imperfections and voids in film deposition.

Patterned films 30 can be created by treating surfaces with ion species that enhance or inhibit neuronal cell attachment allow subsequent proliferation and/or differentiation of the neurons to form desired patterns. Plasma deposition of optically transparent, electrically conducting, ultra-thin metal films can also be used to form electrodes for extra-cellular electrical stimulation of neurons. Mask works can be and were used to form patterns of ion beam or plasma deposition treated regions and thereby promote patterned neuron growth.

Plasma deposition is preferably performed using a filtered vacuum arc system that has been described in detail by Brown I. G., Anders A., Dickinson M. R., MacGill R. A. and Monteiro O. R. (1999) Surf. Coat. Technol. 112, 271; Anders S., Anders A. and Brown I. G. (1993) J. Appl. Phys. 74, 4239; Boxman R. L., Martin P. J. and Sanders D. M. (1995), editors, Vacuum Arc Science and Technology, Noyes, N.Y., which are hereby incorporated by reference in their entirety. A simplified schematic of the filtered vacuum arc plasma deposition system is shown in FIG. 3.

The vacuum arc (or cathodic arc) is a high current discharge between two electrodes in vacuum. Metal or carbon plasma is produced in abundance from the cathode material in the plasma gun, and it is this plasma that carries the arc current. In a preferred embodiment, a repetitively pulsed vacuum arc plasma source is used; the pulse length should be about 5 msec and the repetition rate about 1 pps. Along with the metal plasma that is generated by the vacuum arc, a flux of macroscopic droplets of size in the broad range 0.1-10 microns is also produced, and routine use of a 90° magnetic filter can be used for their removal—a curved ‘plasma duct’ which stops line-of-sight transmission of macroparticles while allowing the transmission of plasma by virtue of an axial magnetic field which ducts the plasma through the filter (Anders A., Anders S. and Brown I. G. (1994) J. Appl. Phys. 75, 4900). The substrate (CCD) should be mounted on a grounded holder positioned about 10 cm from the duct exit.

Referring now to FIG. 3, a suitable apparatus for depositing the thin films used in continuous protective film 20 (aluminum, silica, etc.) and patterned film 30 (diamond like carbon or other inert organic material) is shown schematically. The apparatus illustrated is known in the art. An arc power supply is connected to a plasma gun which comprises an anode located upstream from a cathode so that the applied charge between the electrodes results in a plasma being discharged as illustrated by the arrowed lines. The cathode material is eroded by the arc discharge and converted into a plasma. After filtering droplets of molten material (by a shield/magnetic island or by using a toroidal filter), this plasma is deposited onto the substrate (in this case the CCD surface).

The plasma is directed through the plasma duct, which has coils around it to maintain the charged state of the plasma and to focus it. It is curved to trap macroparticles that tend to travel in a straight line. A vacuum is applied to the plasma duct, and a controlled amount of a selected gas may be introduced into the plasma duct through a gas inlet (not shown). The plasma is deposited onto the substrate, which is electrically grounded. A mask is placed on or just above the substrate to permit deposition only on selected areas when patterning is desired.

A monitor is attached to the substrate to allow routine adjustment of beam voltage and current for the desired deposition characteristics.

Suitable apparatus for forming the present coatings on the CCD surface is also shown in U.S. Pat. No. 4,714,860, and, alternatively, U.S. Pat. No. 4,407,712, hereby incorporated by reference.

By doing the depositions at a somewhat elevated background pressure it is straightforward to form metal oxide films such as, aluminum oxide, titanium oxide and tantalum oxide. A characteristic feature of vacuum-arc-produced plasmas is the relatively high directed energy with which the ions are formed, in the approximate range 20 to 150 eV depending on the ion species (Anders A. and Yushkov G. Yu (2002) J. Appl. Phys. 91, 4824). The patterned film deposition is thus an energetic deposition, which in the preferred embodiment results in the carbon film material formed being high quality, hydrogen-free, DLC films.

E. Insulator 50

In a preferred embodiment, the neuron chip platform 100 has an insulator 50 which can act as a physical and electrical barrier to protect the electrical connection means 60 leading in and out of the CCD. It surrounds the edges and bottom surface of the CCD 10. The insulator 50 can also act as a barrier to keep the cells and cell medium above the CCD 10 and as a sealant to protect the sides and bottom of the CCD from contact with the cell culturing layer 40.

The insulator 50 can be made of any material that will protect the CCD 10 and electrical connection means 60 from corrosion by the typical cell medium. Since the cells will be grown on the platform for extended periods of time, the insulator must be inert and not react with the medium, nor should it degrade and allow the medium to seep through to the CCD circuitry or the electrical connection means 60. Materials that may be useful as insulator materials include but are not limited to, any wire insulator, sealant or bonding agent known in the art for use as a wire insulator, including but not limited to epoxies, wax, parylenes, synthetic rubber or elastomers, polymer compositions having no porosity, glass, ceramic or porcelain, a thin insulating or a polymer/thin film coating a substrate such as a metal and combinations thereof.

It is preferred that the material used for the insulator not be a material with an unknown wettability property because such materials may interact with the salts present in cell medium and it is unclear how long these materials can remain in contact with cell medium. Since the neurons and the protective film 20 and patterned film 30 will remain on the chip and in contact with cell medium for extended periods of time, it is preferred that the insulator bear the same property of inertness and impermeability as the protective and patterned films.

In commercially sold CCDs, the pixel array is often installed in a package or a carrier by the manufacturer such as ceramic or a plastic, complete with wires, pins, and socket pins. The insulator 50 is contemplated to include the package or carrier that the CCD is installed within in some embodiments.

F. Cell Culturing Layer 40

The neuron chip platform 100 is further comprised of a layer of cell culturing materials 40 applied over the patterned film 30 to promote neuron growth and maintain prolonged neuron or cell growth on the platform. It was found that the DLC patterned film 30 in combination with a cell culturing layer 40 produced the best coating for the promotion of nerve growth. Therefore, in a preferred embodiment, a cell culturing layer 40 is added on top of the patterned film 30.

The cell culturing layer 40 is preferably comprised of Collagen Type I, Collagen Type IV, laminin, fibronectin, poly-L-lysine, extracellular basement membrane proteins, growth factors or combinations thereof. In a preferred embodiment, the cell culturing layer 40 is comprised of Collagen Type I protein.

As related in the examples, neurons were cultured on several treated substrates that were coated with Type I Collagen and the growth and differentiation of cells was monitored. Neuron proliferation and the elaboration of dendrites and axons after the addition of nerve growth factor both showed excellent contrast, with prolific growth and differentiation on the treated surfaces and very low growth on the untreated surfaces.

G. Cells 55

Any type of vertebrate or invertebrate neurons can be used. Neuronal cells for the invention can be isolated from various origins including but not limited to, vertebrate or invertebrate species, normal or tumorigenic cells, various tissue origin such as corneal tissue, brain, discarded frontal lobe tissue, spinal tissue and the like.

In other embodiments, neurons of a particular size should be used. For example, pedal ganglia cells from pond snails L. stagnalis may be used in one embodiment because these cells have neuronal cell bodies which can be 30-100 μm in size.

Other factors in choosing which cells to use include the ease of isolation and availability. Neurons from the bovine optic nerve can be isolated from bovine eyes which can be purchased from animals sacrificed for meat production. These eyes have intact optic nerves, from which neurons can be isolated (Huettner, J. and Baughman, R., J. Neurosci 6:3044-3060 (1986); Meyer-Franke, A., Shen, Shiliang, and Barres, B. A., Molec and Cellular Neurosci 14:385-397 (1999)). Neurons can also be dissociated from neonatal rat brains and are relatively easy to grow. Cultured nerve cell lines, such as PC-12 (rat adrenal phechromocytoma, #CRL-1721), are available from the American Type Culture Collection, Rockville, Md. In a preferred embodiment, PC-12 cells are used because data collected from neuron activity can be compared with published baseline measurements on alternative ion-implanted substrates. However, since these cells are of tumorigenic origin, it may be more preferable to use neurons derived from normal tissues.

In a preferred embodiment, the cells used are selected from the group consisting of cultured normal human neuronal cells from discarded human brain tissue, cultured human neuronal cells from discarded human frontal lobe tissue surgically removed for treatment of epilepsy, neurons from the bovine optic nerve, neurons dissociated from neonatal rat brains, snail neurons and frog neurons. In a preferred embodiment, the number of neurons is preferably ˜10⁴-10⁶ per CCD at the outset to study neural networks.

To isolate pure neuron cultures, neurons should be grown from tissue specimens and enzymatically isolated as single cells and distributed on an extracellular matrix derived from the cells and tissue of origin. The cells that proliferate in these cultures may contain glial cells, so cell sorting procedures may be required to allow isolation of pure neuron cultures. These cells should respond reversibly to nerve growth factor (NGF) by differentiation into the neuronal phenotype with extension of neurites. The nerve cells expand neurites in the presence of NGF in culture. Therefore the cells can be used in two culture periods, either the growth of cells in number in a culture medium with serum but without NGF, or the growth of neurite expansion in the presence of NGF. The former is related to the cell attachment and positioning, and the latter to network formation of neurons.

The invention facilitates the measurement of spontaneous and responsive cellular activity of live cells. Cultured neurons, culture medium, and other growth factors can be added on top of the cells and the cell culturing layer. Cells are cultured under standard conditions with media, e.g. as described in Ehrlicher et al., P.N.A.S., 99:16024-16028 (2002). In some embodiments the live neurons are co-cultured with other cells types found in brain or nerve tissue such as glial cells or astrocytes. Various dyes, ions and labels known in the art may be added to the medium.

H. Cell Potential Measurement Apparatus 200

In a preferred embodiment, the above described device is further comprised in a cell potential measurement apparatus 200 (FIG. 2B) comprising (A) a neuron chip platform 100 provided with a charge coupled device detector (CCD) array 10, a thin protective film 20 over the CCD, a thin patterned film 30 to promote neuron growth, and an insulator 50, which can act as a barrier to protect the CCD and the electrical connection means for providing an electrical signal to the CCD and for leading out an electrical signal from the microelectrodes; (B) an illumination source 90, (C) a stimulation signal supply means 70 to be connected to the electrical connection means 60 of the neuron chip platform for providing electrical stimulation to the cells; and (D) a signal or image processing means 80 to be connected to the electrical connection means 60 of the neuron chip platform 100 for processing an output signal or image arising from electrical physiological activities of the cells.

Generally, measurement conducted by means of the above-configured apparatus 200 of this invention is carried out in the following steps. Sample cells are grown on a neuron chip platform 100 for a sufficient amount of time as to facilitate cell attachment and neurite extension. Upon illumination through the illumination source 90, an image of the cells is obtained through the CCD. The thin films allow the cells to grow in very close proximity to the CCD for accurate measurement and monitoring of spontaneous or stimulated action potential activity of individual cells which can be detected by the CCD. The image or output of the CCD is provided to a signal processing means via the electrical connection means connecting the CCD to the signal processing means. The output is then analyzed and recorded or sent to a display device after going through the necessary signal processing.

1. Electrical Connection Means 60 and Signal Processing Means 80

In this invention, the signal detection and analysis of the neural action potentials throughout the large network will use the application of CCD pixel detector arrays 10. Subsequent analysis of the neural array action-potential activity recorded by the detector arrays will follow by signal processing means 80, preferably through computer analysis. This novel approach has the immense advantage of making use of existing technology to provide the large array of pick-up electrodes, associated electronics, and display of the CCD-derived space-time pattern of neural signal activity. One might be able to visualize the complete activity array on a monitor screen, and to record on a hardware or memory device, the activity as a function of time for subsequent analysis.

Neuron action potential activity has previously been recorded using extracellular micro-electrodes (Pine, J., J. Neurosci Methods 2:19-31 (1980)). However, the use of extracellular microelectrodes is fundamentally limited in the number of electrodes that can be employed. This is because each electrode has an individual wire or electrical connection means that transfers the signal to be processed. The instant invention, on the other hand, makes use of the very sophisticated scanning capabilities of CCD pixel detector arrays to allow essentially in excess of a million electrodes because CCDs can typically have 1 to 4 million pixels. This fundamentally changes the depth of detection and measurement that the apparatus of the invention can detect and process, since each pixel can act as an extracellular microelectrode to measure local cellular potential changes.

In a preferred embodiment, the signal processing means 80 would include electronics such as a computer having a digital frame grabber which is connected and controlled by software and hardware to record and capture the digitized pixel values. The digital frame grabber should have a high image capture rate of at least 30 high resolution frames/second. The invention contemplates the use of cooled CCD cameras and available software or hardware for the capture of nerve activity by measuring fluorescence and optical recording of fluorescent calcium gradients in neural tissues as described in Lasser-Ross, N. et al., “High time resolution fluorescence imaging with a CCD camera” Journal of Neuroscience Methods, 36 (1991) 253-61, and hereby incorporated by reference. Examples of other recording systems for use in the signal processing means in the invention are described by Mammano, F., et al., “An optical recording system based on a fast CCD sensor for biological imaging,” Cell Calcium (1999) 25 (2), 115-123; Young, S., Wong, R, and Bianchi, R., “Simultaneous Intracellular Recording and Calcium Imaging in Single Neurons of Hippocampal Slices,” Methods 21, 373-383 (2000); 1; and Potter, S. M., Mart, A. N. and Pine, J. “High-speed CCD movie camera with random pixel selection, for neurobiology research. SPIE Proceedings (1997) 2869: 243-53, and hereby incorporated by reference.

CCD monitoring of the action potential activity will generate massive amount of spatio-temporal events that need to be cataloged and analyzed. The signal processing means 80 may also include an analog-to-digital converter, memory recording devices such as an optical memory disk recorder, and computer hardware or software for analysis that will focus on the process of pattern formation, saliency, and model recovery for inter-cellular communication. The invention also contemplates a system such as BioSig (Parvin, B., et al., IEEE Computer, July 2002; Parvin, B., et al., IEEE Int. Symposium on Bio-Engineering and Computational Biology, November 2000)—a system developed to be used to store and catalog time series events. Algorithms and software can be developed and integrated for subsequent analysis of signals and action potential data collected from the cells.

2. Stimulation Signal Supply Means 70

A stimulation signal can be applied by a stimulation signal supply means 70 via an electrical connection means 60 and measurement and monitoring of cell response to such stimulation signal can be carried out. The stimulation supply means can be electronics, hardware, a power supply, battery, signal supply or generator for generating and applying neuron stimulation signals and/or a computer for measuring, controlling and monitoring the cell response to such signal stimulation.

One approach to a stimulation signal for neuron excitation will be to use extra-cellular electrical stimulation, such as electrodes, through electrically-conducting, optically-transparent, plasma deposited ultra-thin metal films, and the simultaneous recording of self-generated action-potentials of large numbers of neurons throughout the network by novel embodiments of a CCD used for extra-cellular voltage pick-up.

As described above, the invention contemplates protective films 20 which are composite or multiply layered films that consist primarily of combinations of different oxides and/or nitrides. Protective films 20 may have microelectrodes and conducting tracks embedded or buried in the layered film. In such an embodiment, the stimulation signal supplied by the stimulation signal supply means 70 will travel to regions of interest on the CCD to various microelectrodes via conducting tracks.

3. Illumination Source 90

An illumination source 90 could be any external illumination source or an internal illumination source such as when using a backlit CCD as described in U.S. Pat. No. 6,259,085 and is incorporated by reference. The illumination source 90 should provide uniform illumination. Under uniform illumination, the number of electrons in each detector well of the CCD will be a function of the effective area of that well. The areas are quite uniform in a modern chip, but for high quality pictures, the well areas are measured with uniform light and the picture values adjusted to compensate.

If there are small localized voltage differences detected from the neuron fibers by the chip detectors, this will modify the fields defining adjacent wells, and change their areas slightly, increasing one and decreasing its neighbor. With uniform illumination, this should result in a small change in the outputted image. It is expected that the change will be small, and will require subtraction of a before image from an after image to be visible. This subtraction may be done in image enhancing or manipulation software such as Adobe Photoshop or NIH Image software or other similar software.

Alternatively, the illumination source may be a xenon or mercury lamp to enable detection by the CCD of fluorescence. It is well known in the art that CCDs are useful in detecting and observing electrical changes and membrane potentials in neurons through the use of fluorescent calcium indicator dyes or dyes to some other ion or target of interest.

J. Future Applications

The present neuron chip platform 100 has many broad applications including, but not limited to, use as an artificial synapse chip, to create tissue-engineered neurite conduits or as use as biosensors. The present cell potential measurement apparatus 200 is contemplated for use as a research tool to study neurons and synaptic interactions of patterned neuron networks. Large in vitro networks could show, for example, the emergence of stable patterns of activity, and could lead to an understanding of how groups of neurons learn after repeated stimulation.

The neuron chip platform of the invention would permit the study of a large population of neurons, allow modeling of mammalian brains and investigations into neuronal networks, and enable the implementation of novel bio-computer architecture. It is also contemplated that a plurality of neuron chip platforms may be coupled and connected to each other to generate an arrayed platform having approximately the same number and or types of neurons present in a mammalian brain.

The apparatus and methods of the invention also contemplate the use as an artificial synapse chip. Severed or injured nerves in a mammal may be re-connected by an artificial synapse chip comprised of the neuron chip platform of the invention.

### EXAMPLE 1

### Neuron Growth

PC-12 neurons were obtained from the American Type Culture Collection (Manassas, Va.). The PC-12 cell-line was derived from a transplantable rat pheochromocytoma from the adrenal gland. The cells are grown in RPMI 1640 media with 2 gm/L glucose (Invitrogen), 10% heat-inactivated horse serum (Invitrogen), 5% fetal bovine serum (HyClone), 2 mM L-glutamine, 1.5 g/L sodium bicarbonate, pen strep at 37° C., 7.5% CO₂ on Type I Collagen coated Biocoat™ (Becton Dickinson) plastic 100 mm petri plates. Stock cultures were fed every three days with ⅔rds fresh media, and subcultured every 9 days with a 1:4 cell split ratio. Nerve Growth Factor (NGF) 2.5S (Invitrogen) was added to cell densities at concentrations of 50 ng/ml. On a collagen-coated substrate, neurite elongation proceeds at an average rate of ˜50 μm/day for at least 10 days. After 2 weeks of NGF exposure, the cultures generate a dense mat of neuritic processes. Generally, at least 90-95% of the cells in the cultures produce neurites.

The PC-12 cells were inoculated onto pre-cleaned, plasma deposited DLC-coated, Type I Collagen-coated sterile glass slides at 1×10⁵ cells/ml. Cells were allowed to adhere to the slide in a 7.5% CO₂ incubator at 37° C., for 3 hours, and then gently flooded with growth media. Cell growth was monitored by phase light microscopy. Cells were photographed with a digital Spot Camera on a Nikon TMS scope using the Spot Advanced software, and printed using Adobe Photo Shop. PC-12 cells double every 96 hours. After 3-6 days of cell growth, NGF was added to the media at 50 ng/ml. After the addition of NGF, cell division stops and differentiation begins. Cultures were visually monitored daily and images captured every other day, up to 1.5 months after initiation of the cultures.

### EXAMPLE 2

### Plasma Deposition of Various Coatings

Plasma deposition was done using a filtered vacuum arc system as shown in FIG. 3. The vacuum arc (or cathodic arc) is a high current discharge between two electrodes in vacuum. Metal (or carbon) plasma is produced in abundance from the cathode material, and it is this plasma that carries the arc current.

A repetitively pulsed vacuum arc plasma source was used; the pulse length was 5 msec and the repetition rate was 1 pps. Along with the metal plasma that is generated by the vacuum arc a flux of macroscopic droplets of size in the broad range 0.1-10 microns is also produced, and we routinely use a 90° magnetic filter for their removal—a curved ‘magnetic duct’ which stops line-of-sight transmission of macroparticles while allowing the transmission of plasma by virtue of an axial magnetic field which ducts the plasma through the filter (Anders A., Anders S. and Brown I. G. (1994) J. Appl. Phys. 75, 4900). The substrate was mounted on a grounded holder positioned about 10 cm from the duct exit.

Films were in this way formed on the glass microscope slides and CCD chips, of thickness in the approximate range of about 100-300 Å. Ion species used, and thus the kinds of film materials formed, included C, Mg, Ti, Pd, Ta, Ir, Pt and Au. By doing the depositions at a somewhat elevated background pressure it is straightforward to form metal oxides, and thus we also made films of aluminum oxide, titanium oxide and tantalum oxide. A characteristic feature of vacuum-arc-produced plasmas is the relatively high directed energy with which the ions are formed, in the approximate range 20 to 150 eV depending on the ion species (Anders A. and Yushkov G. Yu (2002) J. Appl. Phys. 91, 4824). The film deposition is thus an energetic deposition, and for the case of carbon this results in the film material formed being a high quality, hydrogen-free, diamond-like carbon (DLC) (Pharr G. M., et al., (1996) Appl. Phys. Lett. 68, 779; Monteiro O. R. (1999) Nucl. Instrum. Meth. Phys. Res. B148, 12.), not amorphous carbon or graphite. As described below, it was found that the carbon films were particularly advantageous for enhanced neuron growth.

### EXAMPLE 3

### Effect of Various Plasma Deposited Coatings on Neurite Growth

Neurons grew on all the processed substrates, but there was a wide variation observed in the total number of attached cells and their morphology. Under identical neuron growth conditions for each substrate surface tested, the neuronal cell density attained in the cultures was found to vary over many orders of magnitude for the various processing methods investigated. Processing of the substrate as described herein provides a means of controlling neuron growth.

Ordinary glass microscope slides of dimension 1″×3″ were used as the substrate. Ion implantation was found to be universally poor in its effect for the entire range of parameters explored. The growth rate and the culture cell density at all times during the growth periods were both low compared to the growth of cells on films made by plasma deposition. For example, glass slides that had been ion implanted with carbon under a range of conditions, including at particularly low energy (10 keV) so as to form a carbon profile close to the glass surface, and at relatively high dose (1×10¹⁶ cm⁻²) so as to increase the surface carbon concentration, also yielded unimpressive results. Therefore, further exploration of ion implantation as a tool for enhanced neuron growth was quickly abandoned.

Plasma deposition, on the other hand, was seen to provide significantly enhanced neuron growth for some kinds of film materials (plasma deposition species). It was found that the metals provided a generally positive growth enhancement and that all of the metal oxides were generally negative in their effect. The single film material that stood out as providing vastly enhanced growth was carbon, which when deposited as described above is deposited in the form of hydrogen-free diamond-like carbon, or DLC. Therefore neuron growth on carbon surfaces was investigated in more detail.

Variations in DLC film thickness indicated that a film thickness of about 100-150 Å was near optimum. For thinner films, the neuron “contrast ratio”—ratio of neuron growth density on the DLC-coated region to density on the non-DLC-coated region—was less, and thicker films tended to delaminate from the substrate.

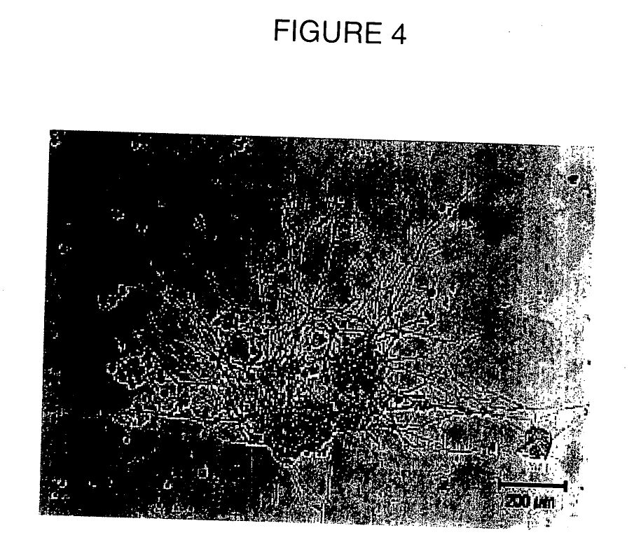

FIG. 4 shows neuron growth after 15 days on a glass slide onto which a 100 Å thick film of DLC was deposited. The DLC region can be seen as a slightly darker region occupying the upper 75% of the whole region viewed; there was no DLC coating on the lower part of the image. The whole slide was coated with Type I Collagen. The photograph in FIG. 4 shows clearly how neurons grew preferentially on a DLC coated substrate. One can see that (i) neuron growth is healthy on the upper DLC-coated region, with virtually no growth on the lower uncoated region, (ii) in the region of good growth, the DLC region, neurons grow extended processes (axons and neurons), and (iii) the neuron extensions show a pronounced tendency to confine their growth to the DLC region.

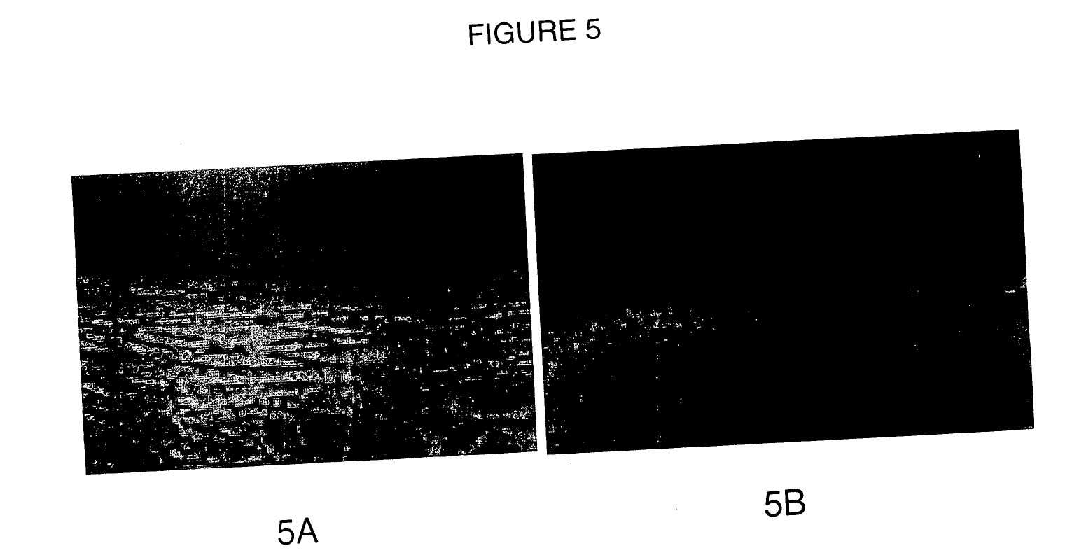

The results of another growth experiment are shown in FIG. 5. Here the neuron density is prolific, much greater than would be chosen for a controlled experiment. But the point is made beautifully clear that the growth is limited to only the DLC-coated region. The lower part of each photograph shown was DLC coated, with the upper part not coated; the substrate was collagen coated, and neurons were then seeded over the entire surface. A delicate neurite growth develops on the DLC-treated region (left-hand photo), which develops into a dense and prolific neuron field (right-hand photo). (Scale: the width of each photograph is about 300 microns).

The plasma deposition was such that the lower part of each photo is the DLC-treated region, and the upper part is not DLC-treated. The entire substrate was collagen coated, and the neurons were seeded over the entire surface. The left-hand photo (FIG. 5A) shows the delicate neurite growth that develops on the DLC-treated region; the right-hand photo shows that the neuron growth in the DLC-treated region continued to a dense and prolific neuron density. These results indicate that neurons grew selectively on the lower DLC-treated regions and not on the upper untreated regions. The contrast or ratio of neuron density in the treated region to neuron density in the untreated region was very high, and neuron growth in the treated region was healthy.

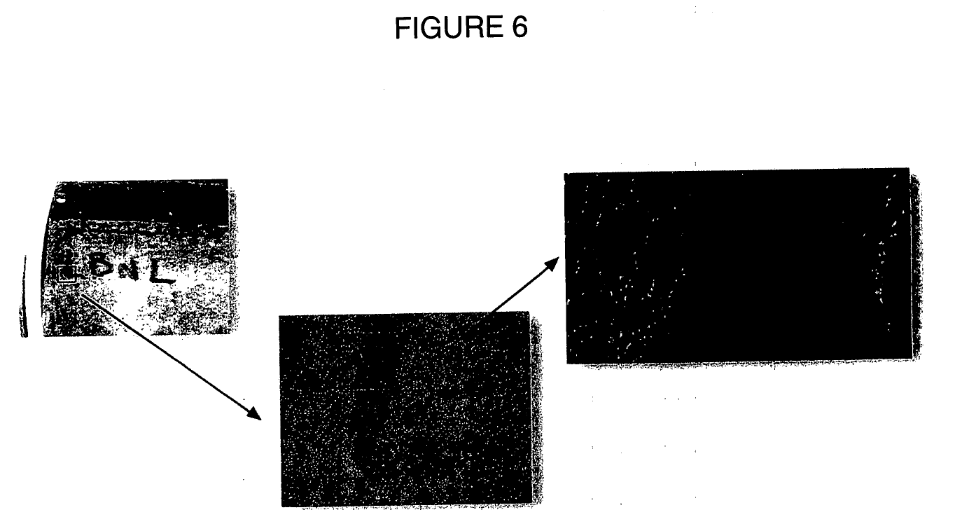

The results of neuron patterning are shown in FIG. 6. Neuron growth is on a glass substrate processed by plasma deposition of ˜150 Å diamond-like carbon (DLC) film. Prior to deposition, “LBNL” was written on the glass slide using a fine marker pen, and then the DLC deposition was carried out. After DLC deposition, the ink was removed with alcohol, thus leaving “LBNL” patterned in the negative in the DLC film. The slide was then coated with Type I Collagen and seeded with PC-12 rat neurons. The neurons were allowed to grow for 3 days, at which point NGF (Nerve Growth Factor) was added. The micrographs shown in FIG. 6 were taken after a growth period of 6 days after initiation of the cultures. Neuron growth was promoted on all areas except on the letters “LBNL” which were not patterned with DLC film.

### EXAMPLE 4

### Patterned Neuron Growth on a CCD

The CCD array chip used in the neuron chip platform was the SONY ICX038DLA, a diagonal 8 mm (Type ½) CCD image sensor, for SONY COHU 112 black-and-white video cameras. The chipboard was removed from the camera to allow easy access to the CCD. The glass cover on the CCD chip was first removed. The surface of the CCD is covered by a plastic layer with a pattern of microlenses over the conductor pattern and around the bonding wire pads. This plastic layer needed to be removed to allow thin films to be deposited on a smooth surface close to the detector array.

Since the chip surface is flooded with cell medium, a thin insulating and protective layer over the chip surface, and insulation over the bonding wires at the chip edge, were needed. Various materials were used as the insulator 50 to protect the bonding wires including epoxy (Hardman 04001 “Double/Bubble” extra fast setting, LBNL stock). To protect the chip surface and the wires on the CCD, a wax coating and a parylene coating of various thicknesses were used. The parylene coating was applied by vapor deposition.

PC12 Neurons were grown according to the method described in Example 1 with the exception that the cells were inoculated onto pre-cleaned, plasma deposited DLC-coated, Type I Collagen-coated sterile CCD chips at 1×10⁵ cells/ml. PC-12 neurons could be kept growing on the CCD chip surfaces for 2 weeks or more. The neuron maintenance on the surface of the chip continued despite the electrolysis of the wires.

The wax and parylene coatings did not insulate the wires on the chip or protect the detectors as desired due to problems encountered with the pre-existing plastic layer and microlenses which the manufacturer deposited on the detector array. Efforts to remove the plastic were not successful because the manufacturer did not disclose the exact composition of the plastic layer. The manufacturer claims that the plastic is an acrylic polystyrene resin, but MEK, acetone, alcohol, ethylene dichloride, and trichloroethylene did not dissolve it. The protective film was able to protect the CCD for up to several days.

Applying an additional coating over the plastic was not successful. The plastic layer becomes jelly-like after a few days in the solution, letting the solution creep under the epoxy to the bonding wires. Electrolysis dissolved the wires, destroying the chip. Preferably a chip with a plastic layer that can hold up to cell medium for an extended period of time should be used, or more preferably the plastic layer needs to be completely removed or protected, or even more preferably, CCD chips without this plastic layer should be obtained.

### EXAMPLE 5

### Patterned Arrays of Neurons on a Neuron Chip Platform

A CCD 10 is obtained from a CCD chip vendor with no plastic covering and having a pixel size of about 6 microns square (6×6) with a chip size of 7.95 mm×6.45 mm, unit cell size of 8.4 μm×9.8 μm and 380,000 total effective pixels. The CCD detectors in the CCD 10 would pick up the electrostatic changes in the cell and process the signal changes through the signal processing means 80. The following film depositions are done under conditions which would permit the deposition of films 20 and 30 that are sufficiently free of imperfections to prevent corrosion or electrolysis of the CCD.

A protective parylene (poly (para-xylene)) film 20 is deposited using an evaporative method to a thickness preferably less than 1000 Angstroms and more preferably about 100 to 500 Angstroms. Alternatively a protective multiply-layered thin film 20 of about 500 Angstroms comprised of silicon dioxide/silicon nitride or silicon dioxide/silicon nitride/silicon dioxide is deposited by filtered vacuum arc plasma deposition on the CCD. The microelectrodes and conducting tracks are embedded or buried in the layered film and applied by plasma deposition. The microelectrodes and conducting tracks are made of silver, platinum, gold, titanium, carbon, silicon or other metal suitable for use as a microelectrode or conducting track. The pattern and for forming the microelectrode array is such that the microelectrodes are placed in regions of interest on the CCD and connected to the voltage source via the conducting tracks and electronic connection means 60.

The electrical connection means 60 and the signal processing means 80 that come with the CCD are protected by an insulator 50 such as epoxy over the wires to seal and insulate from corrosion and electrolysis by the cell culturing medium and also held by the CCD ceramic package to form a stable neuron chip platform.

A patterned film 30 of about 100 to 150 Å diamond-like carbon (DLC) film is then deposited on the protective film 20 according to the desired pattern of neuron growth.

A cell culturing layer 40 of Type I Collagen is then added to the top of the patterned film and seeded with neuron cells 55 such as PC-12 rat neurons. The neurons 55 are grown in the culture medium on the CCD surface in a pattern that is determined by the plasma-deposited DLC thin patterned film 30. The neurons are allowed to grow for 3 days, at which point NGF (Nerve Growth Factor) is added. PC-12 neurons could be kept growing on the CCD chip surfaces for 2 weeks or more to allow the neurons to develop strong dendritic connections and to facilitate cell attachment and neurite extension.

### EXAMPLE 6

### A Cell Potential Measurement Apparatus 200

A cell potential measurement apparatus 200 can be made by providing the neuron chip platform 100 of Example 5, an illumination source 90, a stimulation signal supply means 70 to be connected to the electrical connection means 60 of the neuron chip platform for providing electrical stimulation to the cells for providing an electrical signal to the CCD and for leading out an electrical signal from the CCD or microelectrodes; and a signal or image processing means 80 to be connected to the electrical connection means 60 of the neuron chip platform 100 for processing an output signal or image arising from electrical physiological activities of the cells

The microelectrodes embedded in the protective multiply layered film 20 would be connected to a stimulation means 70 via electrical connection means 60. The stimulation means 70 would provide the appropriate pulse voltage signal to an electrode to generate electrical stimulation of the neurons at selected places on the surface of the CCD chip, preferably around the edge regions. A neuron 55 sitting on top of an electrode should detect the signal because the neuron should be stimulated by the capacitively-induced signal from the electrode. Synaptic transmission may be triggered and the neuron may then pass on the signal to other surrounding neurons through the generation of an action potential. The CCD detectors in the CCD 10 would detect the electrostatic changes in the neurons and process the signal changes through the signal processing means 80.

### EXAMPLE 7

### Patterned Arrays of Neurons on the Neuron Chip Platform and Measurement of Spontaneous and Stimulated Activity

A neuron chip platform of Example 5 and the cell potential measurement apparatus of Example 6 may be used for non-invasive study of live neuron spontaneous activity and activity when stimulated with environmental changes. Effects of such environmental changes including but not limited to, toxins or poisons, temperature and light change, signal or information input or other kind of environmental change, can be added to the culture medium or performed to study the amount and type of neuron response to such changes.

The study of live neuron response and activity conducted by means of the configured apparatus of Example 6 can be carried out in the following steps. Patterned growth of neurons is directed by patterned film 30 of diamond-like carbon on the CCD. The neurons are then washed with fluorescent dyes sensitive to calcium such as CALCIUM GREEN-1 (E3-010) or OREGON GREEN-1 (0-6806) available from Molecular Probes (Eugene, Oreg.). Upon illumination through a xenon or mercury lamp 90, an image of the cells can be obtained through the CCD. The output of the CCD is recorded by signal processing means 80 for subsequent display and analysis. The spatial-temporal activity is provided to a signal processing means 80 via the electrical connection means 60, which is then recorded and outputted, for example, to a display device after going through the necessary signal processing. Stimulation at selected points via stimulation supply means 70 can optionally be introduced via plasma-deposited electrodes as described in Example 6. A stimulation signal can be applied by a stimulation signal supply means 70 via an electrical connection means 60 and measurement and monitoring of cell response to such stimulation signal. Thus, the electrostatic changes brought about by a single cell propagating a nerve signal through membrane depolarization can be measured and recorded in real time.

### EXAMPLE 8

### Observing Signal Propagation with the Neuron Chip

A neuron chip platform of Example 5 and the cell potential measurement apparatus of Example 6 may be used for non-invasive study of signal propagation in a single live neuron. A sciatic nerve is first dissected and obtained from an amphibian such as a frog. The protective myelin sheath is removed by injecting a saline solution into the sciatic nerve. The demyelinated nerve axon will be simply laid across the CCD of the neuron chip platform. The basic approach to feasibility testing will be to stimulate the demyelinated nerve and to monitor the CCD output for signals that reflect the action potential activity in the nerve and to observe CCD signals that accompany nerve activity. An input stimulus will be applied at the distal end of the nerve, and changes in potential differences will be recorded along the proximal side. Upon termination of the non-invasive recording, recordings will also be made under the same conditions, but with the nerve axon impaled with a glass electrode filled with a saturated solution of K₂SO₄ and contacted with a chlorinated silver wire. Cutting the axon prep and recording the break in signaling with distance along the nerve will also verify the validity of the recording, and confirmation of the level of background noise signal. Chemical agents that block or inhibit the neuronal signal can also be added to achieve this confirmation.

### EXAMPLE 9

### Test to Confirm Recording from Patterned Neuronal Networks

There are several technical approaches that could be taken to confirm neuronal signaling between cells optically for comparison with the non-invasive detection of electrical signals using the cell potential measurement apparatus of Example 6. These optical approaches are not the same as the detection concept, rather, the optical approaches are alternative means of detection action potential activity in the nerve. Voltage sensitive dyes can be added to the neuronal network preparation to stain the individual cells in the dark. Illuminating the dyed cells with a light with a specific frequency will result in fluorescence and the voltage-sensitive dye will bleach as evidenced by the loss of fluorescence. A baseline of fluorescence decay must be established using an inverted fluorescent microscope. Transient changes in the local voltage due to neuronal activity from an input signal can be then be visualized by the additional loss of fluorescence measured with time after the signal is administered (see Prinz A. A. and Fromherz P. Effect of neuritic cables on conductance estimates for remote electrical synapses, J. Neurophysiol 89:2215-2224, 2003, which is incorporated by reference).

A second approach to confirm neuronal signaling between cells optically for comparison with the non-invasive detection of electrical signals using the cell potential measurement apparatus of Example 6 would involve the use of monitoring calcium ion fluxes with fluorescent Ca²⁺ ion probes evoked by synaptic stimulation (see Yasuda, R., Nimchinsky, E. A., Scheuss, V, Pologruto, T. A., Oertner, T. G., Sabatini, B. L., and Svoboda K. “Imaging calcium concentration dynamics in small neuronal compartments,” Science 10 Feb 219:15, 2004, which is incorporated by reference).

The examples, methods, procedures, treatments, compounds and films contained herein are meant to exemplify and illustrate the invention and should in no way be seen as limiting the scope of the invention. Changes and modifications in the specifically described embodiments can be carried out without departing from the scope of the invention.

What is claimed is:

1. A neuron chip platform, comprising a charge coupled detector array device (CCD), a thin protective film over the CCD, a thin patterned film applied to the protective film to promote neuron growth, and an insulator material for insulating CCD electronics from a neuron culture.

2. The neuron chip platform of claim 1, wherein the detectors on the CCD are about 6 to 15 microns square.

3. The neuron chip platform of claim 2, wherein the protective film and the patterned film are deposited by plasma deposition.

4. The neuron chip platform of claim 3, wherein said protective film is comprised of a single, composite or multiply-layered thin film of alumina, silica, aluminum silicate, titanium oxide, tantalum oxide, silicon dioxide, aluminum oxide, titanium oxide, tantalum oxide, silicon nitride, aluminum nitride, titanium nitride and tantalum nitride, carbon, Mg, Ti, Pd, Ta, Ir, Pt, Au, parylene and combinations thereof.

5. The neuron chip platform of claim 4, wherein said protective film is about 100 to 1000 Angstroms in thickness.

6. The neuron chip platform of claim 5, wherein said patterned film is comprised of diamond-like carbon.

7. The neuron chip platform of claim 6, wherein the patterned film is about 100 to 300 Angstroms in thickness.

8. The neuron chip platform of claim 7, wherein the insulator material is comprised of any wire insulator, sealant, bonding agent, epoxy, a metal, synthetic rubber or elastomer, a polymer composition having no porosity, glass, ceramic or porcelain.

9. The neuron chip platform of claim 8, further comprising a cell culturing layer above the patterned film.

10. The neuron chip platform of claim 9, wherein said cell culturing layer is selected from the group consisting of Collagen Type I, Collagen Type IV, laminin, extracellular basement membrane proteins or combinations thereof.

11. The neuron chip platform of claim 10, further comprising neurons grown on the cell culturing layer.

12. The neuron chip platform of claim 11, further comprising a means to maintain an environment for culture of the cells on said platform to enable long-term measurement and growth, wherein said environmental maintenance means comprising a temperature adjustment means for maintaining a constant temperature, a means for circulating a culture solution, a means for supplying a mixed gas of air and carbon dioxide, and a covering means to keep the cells enclosed on the platform.

13. The neuron chip platform of claim 12, further comprising microelectrodes and conducting tracks deposited on the protective film by plasma deposition.

14. A cell potential measurement apparatus comprising
a. a neuron chip platform comprising a charge coupled detector device array (CCD), a thin protective film over the CCD, a thin patterned film to promote neuron growth, and an insulator;
b. an electrical connection means connected to the neuron chip platform;
c. an illumination source;
d. a stimulation signal supply means to be connected to the electrical connection means of the neuron chip platform for providing electrical stimulation to the cells; and
e. a signal or image processing means to be connected to the electrical connection means of the neuron chip platform for processing an output signal or image arising from electrical physiological activities of the cells.

15. The cell potential measurement apparatus of claim 14, wherein the neuron chip platform is detachable from the electrical connection means, the stimulation signal supply means and the signal or image processing means.

16. The cell potential measurement apparatus of claim 15, further comprising microelectrodes and conducting tracks deposited onto the protective film by plasma deposition.

17. The cell potential measurement apparatus of claim 16, further comprising a cell culturing layer above the patterned film.

18. The cell potential measurement apparatus of claim 17, wherein said cell culturing layer is comprised of Collagen Type I.

19. The cell potential measurement apparatus of claim 18, further comprising neurons grown on the cell culturing layer.

20. A method of detecting and monitoring live networks of neurons comprising the steps of:
a. providing a cell potential measurement apparatus of claim 17;
b. adding a cell culturing layer seeded with neurons;
c. allowing the neurons to develop neurite extensions or dendritic connections;
d. providing electrical or environmental stimulation to the neurons;
e. detecting and recording the neurons response via the CCD;
f. analyzing said neuron response using a signal or image processing means.
